(19) United States
(12) Patent Application Publication
(10) Pub. No.: US 2004/0106849 A1
(43) Pub. Date: Jun. 3, 2004

(54) Multi-functional, bi-directional communication telemetry capsule

(76) Inventors: Jin-Ho Cho, Daegu-City (KR); Hyun-Chul Choi, Daegu-City (KR); Jun-Rim Choi, Daegu-City (KR)

Correspondence Address: NIXON & VANDERHYE P.C., 8th Floor, 1100 North Glebe Rd., Arlington, VA, 22201-4714, US

(21) Appl. No.: 10/308,028

(22) Filed: Dec. 3, 2002

(51) Int. Cl. A61B 1/00

(52) U.S. Cl. 600/101

(57) ABSTRACT

A multi-functional, bi-directional communication telemetry capsule for endoscopy is provided. The multi-functional, bi-directional communication telemetry capsule includes a capsule body insertable into a patient's body, a lens mounted on the capsule body, an imaging sensor installed in the capsule body to image a site of the patient's body viewed through the lens, a pH sensor, a biological potential sensing electrode, a stimulating electrode, a pressure sensor, a light emitter to emit light in front of the capsule body, the light intensity of the light emitter being externally controllable, a transmitter to wirelessly transmit an image signal generated by the imaging sensor, a pH signal generated by the pH sensor, a biological signal generated by the biological potential sensing electrode, and a pressure signal generated by the pressure sensor, a receiver to wirelessly receives an external control signal, and a control unit and a power source for the forgoing constitutional elements.

### BACKGROUND OF THE INVENTION

1. Field of the Invention

The present invention relates to a multi-functional,al, bi-directional telemetry capsule, and more particularly, to a multi-functional, bi-directional telemetry capsule for endoscopy, which is externally controllable and capable of wirelessly transmitting a signal according to an internal state of a patient's body to the outside.

2. Description of the Related Art

An endoscope is a medical instrument widely used for examining visually the interior of a patient's bodily canal or organ such as the stomach, bladder, or colon. However, when an endoscope is inserted via a patient's esophagus or anus and advanced into the small or large intestine, the patient feels so much pain that the insertion itself is difficult and the interior of the organ cannot be visualized.

Furthermore, the intestinal state of a patient cannot be examined with 100% accuracy using conventional endoscopes. For example, an abnormal gastric pH or pressure level cannot be visualized with the conventional endoscope, and the inserted endoscope body is not externally controllable, thereby obstructing an accurate diagnostic examination.

### SUMMARY OF THE INVENTION

Accordingly, the invention provides a multi-functional, bi-directional communication telemetry capsule for a more accurate and easy endoscopic examination, which is externally controllable after being inserted into a patient's body, capable of wirelessly transmitting an internal state of the patient's body, and allows a user to properly visualize a desired organ.

The invention also provides a multi-functional, bi-directional communication telemetry capsule for endoscopy which can measure a pH or pressure level in a patient's organ and can be moved in the body by electrical stimulation.

In an aspect, the invention provides a multi-functional, bi-directional communication telemetry capsule for endoscopy, the capsule comprising; a capsule body insertable into a patient's body; a lens mounted on the capsule body; an imaging sensor installed in the capsule body to image a site of the patient's body viewed through the lens; a light emitter to emit light in front of the capsule body; a transmitter to wirelessly transmit an image signal generated by the imaging sensor; a receiver to wirelessly receive an external control signal; a control unit to control the imaging sensor, the light emitter, the transmitter, and the receiver; and a power source to supply power to the forgoing constitutional elements.

In the multi-functional, bi-directional communication telemetry capsule according to the present invention, the light emitter may include at least one of a white-light light emitting diode emitting white light and an infrared ray emitting diode emitting infrared rays. The light intensity of the light emitter is externally controllable.

The multi-functional, bi-directional communication telemetry capsule according to the present invention may further comprises a pH sensor mounted on the external surface of the capsule body and connected to the control unit to measure a pH level of a site of the patient's body. The multi-functional, bi-directional communication telemetry capsule may further comprise a biological potential sensing electrode mounted on the external surface of the capsule body and connected to the control unit to measure an electromyogram and/or an electrocardiogram. In these embodiments, the pH sensor and the biological potential sensing electrodes may be implemented with ion sensitive field effect transistors (ISFETs) that have a small volume and need no electrolyte for measurement.

The multi-functional, bi-directional communication telemetry capsule according to the present invention may further comprise a stimulating electrode mounted on the external surface of the capsule body and connected to the control unit to apply an electrical stimulus to the intestine. The multi-functional, bi-directional communication telemetry capsule may further comprise a pressure sensor mounted on the external surface of the capsule body and connected to the control unit to measure the inner pressure of a site of the patient's body.

### BRIEF DESCRIPTION OF THE DRAWINGS

The above features and advantages of the present invention will become more apparent by describing in detail exemplary embodiments thereof with reference to the attached drawings in which:

FIG. 1 is a perspective view of a multi-functional, bi-directional communication telemetry capsule for endoscopy according to an embodiment of the present invention;

FIG. 2 is a longitudinal sectional view of the multi-functional, bi-directional communication telemetry capsule in FIG. 1;

FIG. 3 is a block diagram of the multi-functional, bi-directional communication telemetry capsule in FIGS. 1 and 2; and

FIG. 4 is a block diagram of a receiver in FIG. 3.

### DETAILED DESCRIPTION OF THE INVENTION

Embodiments of a multi-functional, bi-directional communication telemetry capsule for endoscopy according to the present invention will be described with reference to the appended drawings.

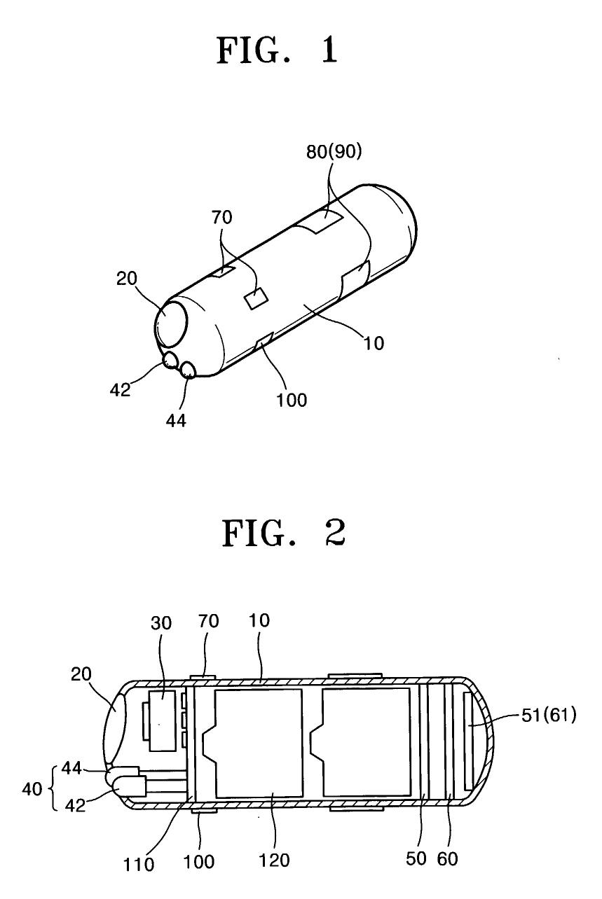

A multi-functional, bi-directional communication telemetry capsule according to an embodiment of the present invention is shown in FIG. 1. FIG. 2 is a longitudinal sectional view of the multi-functional, bi-directional communication telemetry capsule of FIG. 1. Referring to FIGS. 1 and 2, a multi-functional, bi-directional communication telemetry capsule according to the present invention includes a capsule body 10 insertable into a patient's body, a lens 20 mounted on the capsule body 10, an imaging sensor 30 installed in the capsule body 10 to form an image of a site of the patient's body through the lens 20, a light emitter 40 to emit light in front of the capsule body 10, a transmitter 50 to wirelessly transmit an image signal generated by the imaging sensor 30, a receiver 60 to wirelessly receive an external control signal, a pH sensor 70 to measure a pH level of a site of the patient's body, a biological potential sensing electrode 80 to measure an electromyogram (EMG) of the intestine or an electrocardiogram (ECG) of the heart, an stimulating electrode 90 to apply an electrical stimulus to the intestine, and a pressure sensor 100 to measure a pressure level of a site of the patient's body. The multi-functional, bi-directional communication telemetry capsule according to the present invention includes a control unit 110 to control the imaging sensor 30, the light emitter 40, the transmitter 50, the receiver 60, the pH sensor 70, the biological potential sensing electrode 80, the stimulating electrode 90, and the pressure sensor 100, and a power source 120 to supply power to the above-listed elements of the capsule. Antennas 51 and 61, which may be implemented with known loop-coil or spiral antennas, are installed in the capsule body 10 for enhancing wireless transmission and reception efficiency of the transmitter 50 and the receptor 60, respectively. Alternatively, either one of the antennas 51 and 61 may be shared by the transmitter 50 and the receptor 60.

The capsule body 10 has a cylindrical shape of a length of 3 cm or less and a diameter of 0.7-1 cm so as to be insertable into the patient's body and to easily travel through the intestine.

The lens 20 focuses light to allow the imaging sensor 30 to form an image of the interior of the intestine. The lens 20 may be contaminated with bodily mucus. Accordingly, it is preferable that the lens 20 be equipped with a wiper (not shown) for removing the bodily mucus adhering to the lens 20.

The imaging sensor 30 that enables a color image to be captured may be implemented with a known charge coupled device (CCD) or a complementary metal oxide silicon (CMOS) semiconductor device.

The light emitter 40 includes at least one of a white-light light emitting diode (LED) 42 emitting white light and an infrared LED 44 emitting infrared rays. The white-light LED emits white light to illuminate a dark lumen to allow the imaging sensor 30 to form a color image. At this time, when a tumous or inflammantory pathology is in progress in the intestinal wall, the pathological tissue cannot be visualized with the imaging sensor 30. In this case, pathological conditions can be examined by infrared irradiation from the infrared LED 44 based on a temperature difference between normal and pathological tissues. According to the present invention, the on/off state of the imaging sensor 30 and the light emitter 40 is selectively controlled by an external signal so as to reduce unnecessary power consumption. Also, the light intensity of the light emitter can be externally controlled.

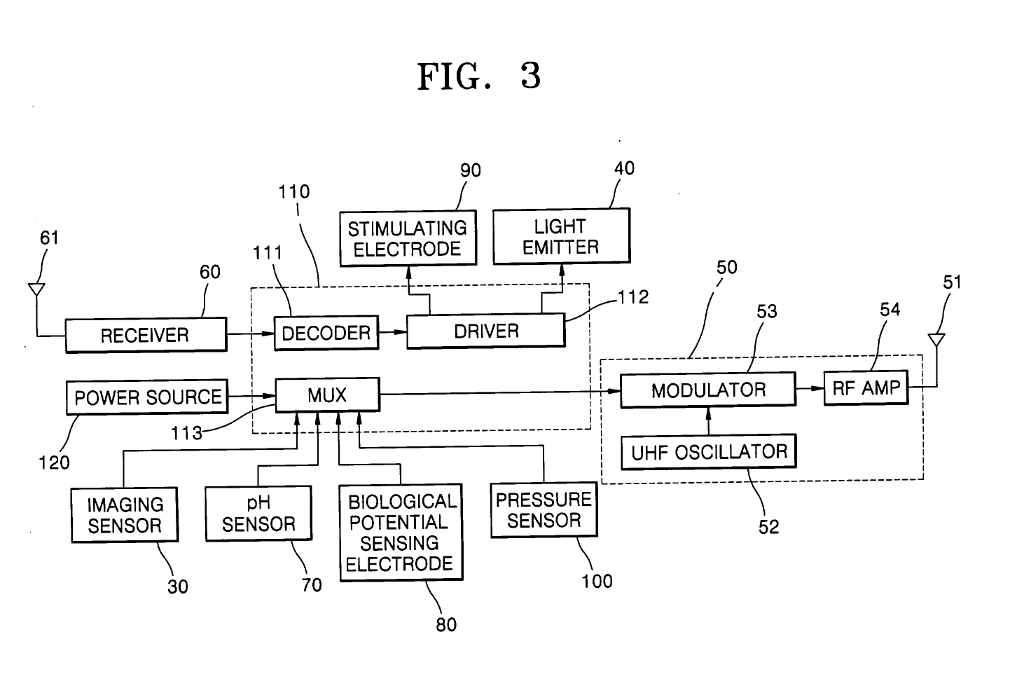

Referring to FIG. 3, which is a block diagram of the multi-functional, bi-directional communication telemetry capsule according to the present invention, the transmitter 50 includes a ultra high frequency (UHF) oscillator 52, a modulator 53, and a radio frequency amplifier (RF AMP) 54. The transmitter 50 modulates the information multiplexed by and transmitted from a multiplexer (MUX) 113 of the control unit 110, which is acquired by the imaging sensor 30, the pH sensor 70, the biological potential sensing electrode 80, and/or the pressure sensor 100, in an UHF range and amplifies and transmits the modulated information. Since the multi-functional, bi-directional communication telemetry capsule according to the present invention has a small size, only a small antenna is needed for the multi-functional, bi-directional communication telemetry capsule. For a high information transmission efficiency when using the small antenna, the transmitter 50 works in a UHF range. If the frequency range used in the transmitter 50 is too high, the image signal may degrade due to frequency absorption and dispersion in the patient's body. Therefore, a frequency range of 300-2400 MHz is suitable for the transmitter 50.

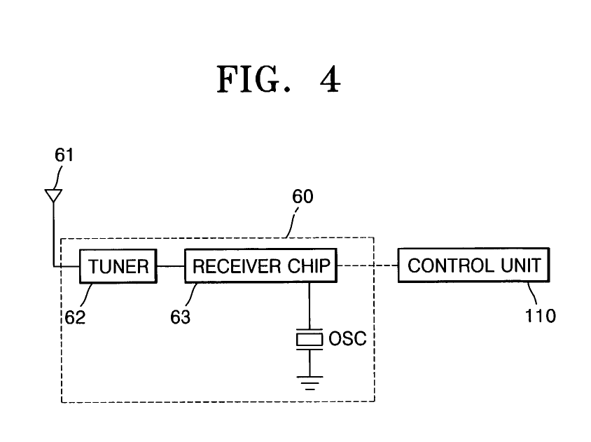

FIG. 4 is a block diagram of the receiver 60 in FIG. 3. Referring to FIG. 4, the receiver 60 includes a tuner 62 and a receiver chip 63. The tuner 62, connected to the receiver antenna 61, is an inductor-capacitor (LC) tank circuit tuned on a UHF frequency transmitted from an external transmitter (not shown). The receiver chip 63 may be implemented with one chip On-Off Keyed (OOK) SMD heterodine receiver chip (MICRF 007, available from MICREL Co.). This 8-pin OOK SMD chip occupies a small area of 4 mm by 6 mm. Since an OOK output signal can be modulated up to 3 kHz, various loads in the telemetry capsule can be controlled using the OOK SMD chip at a frequency of 375 Hz for each channel.

Any frequency band, for example, VHF or UHF band, may be selectively used for the receiver 60. This is because the amount of information carried by the external control signal to the receiver 60 is incomparably less than that of the image signal to be transmitted by the transmitter 50. For example, frequency values of 27.125 MHz, 315 MHz, 433 MHz, etc., available to the public may be used for the receiver 60. A known OOK scheme is suitable for the communication and modulation scheme in the receiver 60.

The pH sensor 70 is mounted on the external surface of the capsule body 10 in order to measure the pH of a site of the patient's body. A smaller pH sensor is more preferred due to the small size of the capsule body 10. For example, an ion sensitive field effect transistor (ISFEF) known for its small volume is suitable for the pH sensor 70. The use of the ISFEF is also convenient because it does not require electrolytes, such as KCl solution, unlike general pH sensors. The pH sensor 70 is very useful to measure the degree of gastric juice secretion or digestion stages.

The biological potential sensing electrode 80 is mounted on the external surface of the capsule body 10 to measure the intestinal EMG or ECG. The EMG acquired by the biological potential sensing electrode 80 provides information on the contractility of the intestine, and the ECG acquired by the biological potential sensing electrode 80 enables a more precise cardiac diagnosis because the cardiac potential is measured in the patient's body.

The stimulating electrode 90 is mounted on the external surface of the capsule body 10 to apply an electrical stimulus to the intestine. When the stimulating electrode 90 applies an electrical stimulus to the intestine, the intestine is forced to move so as to facilitate motion of the multi-functional, bi-directional communication telemetry capsule along the intestine to be excreted through the anus if necessary. The duration for which the capsule stays and operates in the intestine can be controlled by the intensity of electrical stimuli applied to the stimulating electrode 90.

Although in the above-described embodiment the biological potential sensing electrode 80 and the stimulating electrode 90 are described as being separately constructed for the convenience of explanation, it will be appreciated that the bipotential sensing electrode 80 and the stimulating electrode 90 can be constructed as a single electrode.

The pressure sensor 100 is mounted on the external surface of the capsule body 10 to measure the internal pressure of the intestine as an index of a state of an intestinal movement.

The control unit 110 includes a decoder 111 to select an appropriate channel signal among a plurality of channel signals received from the receiver 60, a driver 112 to supply power to the stimulating electrode 90 or the light emitter 40 according to the diagnostic purpose, and the MUX 113 to multiplex a plurality of signals input from the imaging sensor 30, the pH sensor 70, the biological potential sensing electrode 80, and the pressure sensor 100. The control unit 100 is connected to the imaging sensor 30, the light emitter 40, the transmitter 50, the receiver 60, the pH sensor 70, the biological potential sensing electrode 80, the stimulating electrode 90, and the pressure sensor 100 and systematically control the operations of these elements using an external remote signal.

The operation of the multi-functional, bi-directional communication telemetry capsule according to the present invention having the structure as described above will be described.

As a patent swallows the multi-functional, bi-directional communication telemetry capsule through the esophagus, the capsule travels inside the patient's body. When the capsule reaches a predetermined site to be examined in the patient's body, the controller 110 is controlled by an external wireless signal in order to operate the capsule.

For example, the interior of the stomach can be simply visualized by the imaging sensor 30 in cooperation with the white-light LED 42. Whether or not the stomach wall has a pathology in progress can be observed using the infrared rays emitted from the infrared LED 44 and imaged by the imaging sensor 30. For a more precision gastric condition examination, pH and pressure levels in the stomach can be measured by operating the pH sensor 70 and the pressure sensor 100, respectively, and physiological signals, such as EMG and ECG, can be detected by operating the biological potential sensing electrode 80. The resulting image signal or measured signals are externally transmitted with a ultra high frequency through the transmitter 50.

When there is a need to move the multi-functional, bi-directional telemetry capsule to another site, an electrical pulse is applied to the stimulating electrode 90 located on the external surface of the capsule body 10 in order to facilitate pyloric movement. The multi-functional, bi-directional telemetry capsule according to the present invention can be moved to a target site to be diagnosed by the pyloric movement or can be excreted through the anus if necessary.

Alternatively, electrical stimulation by the stimulating electrode 90 may be applied for therapeutic purposes, for example, in order to alleviate indigestion, constipation, etc.

As described above, the multi-functional, bi-directional communication telemetry capsule according to the present invention can visualize the internal state of a patient's body and can measure pH and pressure levels in a site of the patient's body, thereby enabling a more accurate diagnosis and treatment. The motion of the multi-functional, bi-directional communication telemetry capsule in the patient's intestine can be controlled by electrical stimulation, so that the capsule reaches a target site to be examined. The multi-functional, bi-directional communication telemetry capsule can be timely turned on and off by external control, thereby minimizing power consumption.

While the present invention has been particularly shown and described with reference to exemplary embodiments thereof, it will be understood by those of ordinary skill in the art that various changes in form and details may be made therein without departing from the spirit and scope of the present invention as defined by the following claims.

What is claimed is:

1. A multi-functional, bi-directional communication telemetry capsule for endodscopy, the capsule comprising;
a capsule body insertable into a patient's body;
a lens mounted on the capsule body;
an imaging sensor installed in the capsule body to image a site of the patient's body viewed through the lens;
a light emitter to emit light in front of the capsule body;
a transmitter to wirelessly transmit an image signal generated by the imaging sensor;
a receiver to wirelessly receive an external control signal;
a control unit to control the imaging sensor, the light emitter, the transmitter, and the receiver; and
a power source to supply power to the forgoing constitutional elements.

2. The multi-functional, bi-directional communication telemetry capsule of claim 1, wherein the light emitter includes at least one of a white-light light emitting diode emitting white light and an infrared ray emitting diode emitting infrared rays.

3. The multi-functional, bi-directional communication telemetry capsule of claim 1, wherein the light intensity of the light emitter is externally controllable.

4. The multi-functional, bi-directional communication telemetry capsule of claim 1, further comprising a pH sensor mounted on the external surface of the capsule body and connected to the control unit to measure a pH level of a site of the patient's body.

5. The multi-functional, bi-directional communication telemetry capsule of claim 1, further comprising a biological potential sensing electrode mounted on the external surface of the capsule body and connected to the control unit to measure an electromyogram and/or an electrocardiogram.

6. The multi-functional, bi-directional communication telemetry capsule of either one of claims 4 and 5, wherein the pH sensor and the biological potential sensing electrodes are implemented with ion sensitive field effect transistors (ISFETS) that have a small volume and need no electrolyte for measurement.

7. The multi-functional, bi-directional communication telemetry capsule of claim 1, further comprising a stimulating electrode mounted on the external surface of the capsule body and connected to the control unit to apply an electrical stimulus to the intestine.

8. The multi-functional, bi-directional communication telemetry capsule of claim 1, further comprising a pressure sensor mounted on the external surface of the capsule body and connected to the control unit to measure the inner pressure of a site of the patient's body.
